(19) United States
(12) Patent Application Publication
(10) Pub. No.: US 2026/0232518 A1
(43) Pub. Date: Aug. 13, 2026

(54) LIMB-ASSISTIVE DEVICE WITH MACHINE LEARNING

(71) Applicant: The Regents of the University of Michigan, Ann Arbor, MI (US)

(72) Inventors: Roberto Manuel Leonardo MEDRANO, Ann Arbor, MI (US); Gray C. THOMAS, Ann Arbor, MI (US); Robert D. GREGG, IV, Ann Arbor, MI (US); Elliott J. ROUSE, Ann Arbor, MI (US)

(21) Appl. No.: 19/160,801

(22) PCT Filed: Feb. 28, 2024
(86) PCT No.: PCT/US2024/017689

§ 371 Date: Aug. 28, 2025

Related U.S. Application Data
(60) Provisional application No. 63/448,763, filed on Feb. 28, 2023.

(51) Int. Cl. A61H 3/00; A61H 1/02; G16H 40/67

(52) U.S. Cl. CPC A61H 3/00; A61H 1/0266; G16H 40/67; A61H 2201/5007; A61H 2230/625

(57) ABSTRACT

A powered limb-assistive device includes a controller that uses a neural network to generate an initial prediction of the current gait state of a user of the device based on kinematic measurements from the device. The controller filters the initial prediction to arrive at a current prediction of the current gait state based at least in part on a continually updated measure of trust in the kinematic measurements. The controller can be employed in a powered prosthesis or orthosis, such as a powered exoskeleton that assists user movement of a first portion of their leg relative to a second portion of their leg about a knee and/or ankle joint.

This invention was made with government support under HD094772 awarded by the National Institutes of Health. The government has certain rights in the invention.

### TECHNICAL FIELD

This disclosure is related to powered limb-assistive devices and control strategies intended to improve their function with varying user tasks.

### BACKGROUND

Robotic exoskeletons may someday allow human users to overcome the limitations of our natural bodies. For example, emerging lower-limb exoskeletons can provide assistive joint torques to help a user walk and carry loads with promising outcomes, including reduced metabolic cost. Most research to date has focused on steady-state locomotion in a controlled laboratory setting where the task and phase rate (i.e., rate of continuous progression through the gait cycle) are nearly constant. This controlled environment makes it easier to design control strategies that deliver appropriate torque assistance in synchrony with the user's gait because phase progression during steady-state locomotion can be reasonably predicted using time normalized by the stride period—e.g., the time between consecutive heel strike (HS) events. This approach is quite effective and widely used for controlling exoskeletons on treadmills. However, control strategies based on these assumptions perform poorly outside of the laboratory, where environments are uncertain and locomotion is highly non-steady and transitory. More recent research has focused on development of control strategies that detect changes in human gait throughout different walking conditions, such as changes in walking speed and/or changes in ground incline, which can then be used to adjust the torque assistance from the exoskeletons according to the task.

### SUMMARY

Embodiments of a powered limb-assistive device include a controller that uses a neural network to generate an initial prediction of a current gait state of a user of the device based on kinematic measurements from the device. The controller filters the initial prediction to arrive at a current prediction of the current gait state of the user based at least in part on a continually updated measure of trust in the kinematic measurements.

In various embodiments, the neural network includes a transformers machine learning model.

In various embodiments, the neural network has learned a relationship between the kinematic measurements and human gait states based at least in part on data obtained from human subjects other than the user.

In various embodiments, the learned the relationship is based at least in part on data obtained from the device while the user is using the device.

In various embodiments, the controller uses a Bayesian filter that generates an internal initial prediction of the current gait state based at least in part on a previous gait state prediction of the filter and updates the internal initial prediction to generate the current prediction of the current gait state of the user.

In various embodiments, the measure of trust is generated by another neural network that has learned a heteroscedastic covariance model that encodes prior knowledge about gait state parameters. In various embodiments, the controller uses another neural network comprising a data-driven gait model used to generate a prediction of kinematic measurements for comparison with the kinematic measurements from the device.

In various embodiments, the gait state is a multiparameter vector comprising at least some of the following parameters: gait phase, walking speed, ground incline, a stair locomotion parameter, a stopping parameter.

In various embodiments, the device is a powered exoskeleton including a joint and an actuator that applies a controlled torque at the joint based on an output of the controller.

In various embodiments, the device is a powered exoskeleton including a leg joint and an actuator that applies a controlled torque at the leg joint based on an output of the controller. The exoskeleton assists user movement of a first portion of the leg relative to a second portion of the leg about the leg joint.

In various embodiments, the device is a powered exoskeleton including a knee joint or an ankle joint and an actuator that applies a controlled torque at the joint based on an output of the controller. The exoskeleton assists movement of a lower leg of the user relative to an upper leg of the user about the knee joint and/or assists movement of a foot of the user relative to the lower leg of the user about the ankle joint.

It is contemplated that any one or more of the above-listed features, the below-described features, and/or features disclosed in the drawings can be combined in any technically feasible combination to define a claimed invention.

### BRIEF DESCRIPTION OF DRAWINGS

FIG. 1 is a schematic representation of a limb-assistive device including a controller with an illustrative control architecture; and

FIG. 2 is a schematic representation of the controller illustrating an example of the filter of FIG. 1.

### DESCRIPTION OF EMBODIMENTS

Described below is a limb-assistive device and device controller with a control architecture that learns gait patterns of a user as encoded by their gait state (phase, speed, ground incline, and stair locomotion). The architecture uses an artificial neural network to provide an initial prediction of gait state, which may include gait phase, based on kinematic measurements provided by the device. The architecture also uses a filter designed to generate a filtered prediction based on the initial gait state prediction to assess the reliability of the prediction and selectively apply the filtered prediction to a torque profile used to control joint torque at the device.

The artificial neural network may be a “transformers” machine learning (ML) architecture that employs a feed forward, sequence-to-sequence (seq-2-seq) model and that features an attention mechanism, which is described by Vaswani et al. (“Attention is all you need.” Advances in neural information processing systems (NIPS 2017)). In the context of kinematics and gait state encoding and decoding, the transformers architecture is drawn to capture differences across locomotion types through embedding kinematic measurements into a high-dimensional space that reveals contextual relationships or associations (attention) amongst the kinematic measurements over time and/or across sensor types.

As used herein, the term “limb-assistive device” encompasses both prosthetic and orthotic devices configured to provide or assist movement of a limb or portion of a limb about a natural or artificial joint. “Powered” or “active” devices are distinguished from passive devices in that their behavior is changeable via application of non-user forces or energy to structural device components. The example disclosed below is categorized as a powered orthotic device in the form of an exoskeleton that assists the user with movement of a first portion of their leg relative to a second portion of their leg about a joint. Specifically, the first portion is a foot portion, the second portion is a lower leg portion, and the joint is an ankle joint. In another embodiment, the first portion is a lower leg portion, the second portion is an upper leg portion, and the joint is a knee joint. In another embodiment, the device is an exoskeleton that assists the user with movement of their upper leg portion relative to their torso about a hip joint. Various embodiments are assistive devices assisting movement about multiple joints, such as a knee-ankle orthosis, a hip-knee orthosis, or a hip-knee-ankle orthosis. The disclosed control strategy is applicable to prediction of the state of other human motions as used to control assistive device movement about a joint.

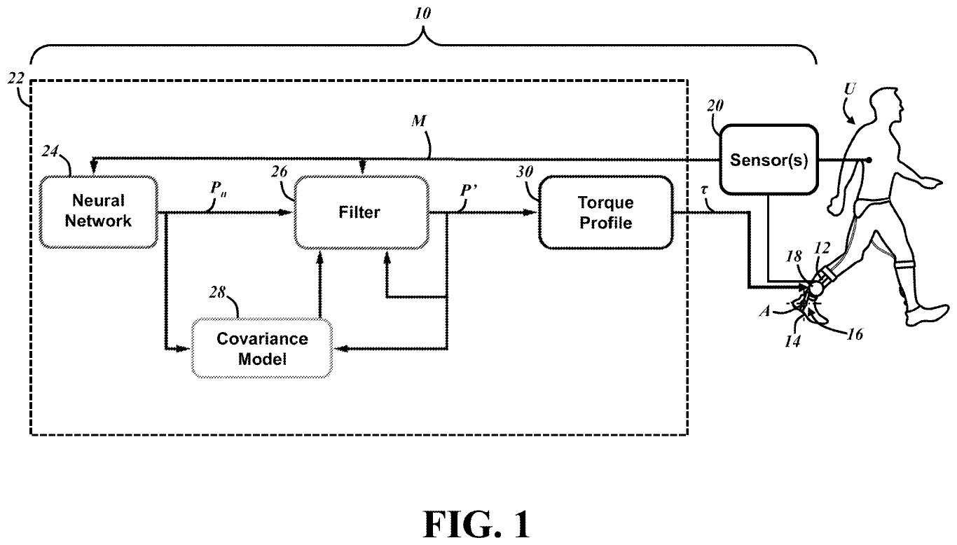

FIG. 1 is a schematic representation of a limb-assistive device 10. The illustrated example is a powered ankle exoskeleton 10 configured to assist movement about an ankle joint of a user U. The exoskeleton 10 includes first and second structural members 12, 14 coupled at a joint 16 that provides rotational movement of the structural members 12, 14 relative to each other at least about an axis A. In this case, the first structural member 12 is a leg brace configured for attachment to the lower leg between the knee and ankle of the user U, and the second structural member 14 is a foot plate configured to fit along and move with the user's foot, which may be fitted along a shoe sole as shown. The device 10 additionally includes an actuator 18, such as an electric motor, that provides a torque t at the joint to change the relative rotational position of the leg brace 12 and foot plate 14 or to impede changes in the relative rotational position of the structural members 12, 14. The device 10 additionally includes one or more sensors 20 and a controller 22. A sensor 20 is any feature of the device that produces or collects information pertinent to user movement, such as bodily movements during a walking gait cycle. The controller 22 receives information from the sensor(s) 20, processes the information, and controls the actuator 18 based on the processed information.

In this example, the one or more sensors 20 includes a sensor that detects an angle defined between the leg brace 12 and foot plate 14 relative to a reference angle and may be embodied as an encoder associated with the actuator 18. Another type of sensor 20 that provides information pertinent to user gait is a heel strike sensor. Each sensor 20 may be affixed to one of the structural members 12, 14, the joint 16, or some other component of the device 10. In some cases, a sensor 20 may be physically separate from the remainder of the device 10 and affixed to the user U elsewhere along their body, such as an accelerometer in communication with the controller 22 that detects transverse rocking movement of the user's torso during walking. The device may include other unillustrated components, such as a transmission coupling the actuator with one of the structural members 12, 14, a spring and/or damping system, and a portable power source (e.g., a battery).

FIG. 1 includes a simplified schematic of the architecture of the controller 22, which is configured to control output torque τ of the actuator 18 based on information received from the sensor(s) 20 and processed by the controller. The controller architecture leverages machine learning for prediction combined with filtering to improve robustness. The illustrated architecture employs a neural network 24 that can learn the relationship between a gait state and kinematics using only labeled data, which eliminates the difficult task of attempting to fit an explicit mathematical model that describes that relationship. However, because of the opaque “black box” nature of machine learning models—i.e., the lack of this same explicit model—it can be difficult to discern why the network made a specific prediction. A prediction filter 26 is thus employed to smooth the predictions generated by the neural network 24 and update each prediction based on the previous filtered prediction and information encoded about the gait state in the sensor measurements, as described further below. The controller 22 also employs at least one covariance model 28, which operates to determine discrepancies between predicted and measured kinematics and gait state to provide the filter with information on the level of trust to assign to the kinematic and gait state measurements, and a torque profile 30, which is used to convert the filtered prediction P′ of the current gait state to the output torque τ to be applied at the device 10 by the actuator 18.

The primary neural network 24 may use the Transformer architecture to learn and thereby encode kinematic measurements related to human gait cycles and, in use, decode kinematic measurements M generated by the device sensor(s) 20 into a succinct gait state vector including, for example, gait phase, walking speed, ground incline, stair locomotion, and/or transient motions, such as starting or stopping motions. The neural network 24 continually yields initial predictions Pₙ of the current gait state based on the kinematic measurements M provided by the sensor(s) 20. Additionally, the neural network 24 is fine-tunable with individualized data and can thus be personalized to individual users, which is critical given that people can vary significantly in their individual gait patterns.

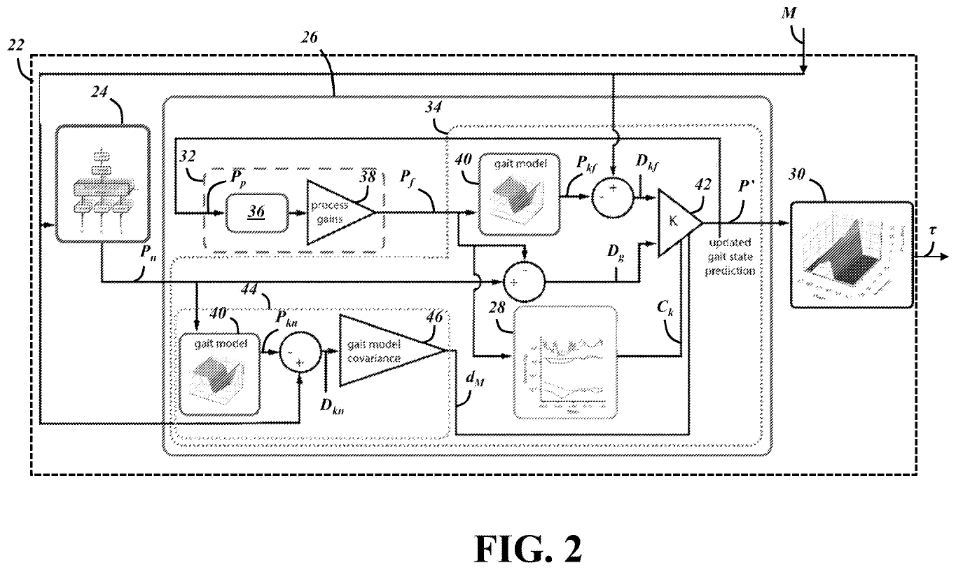

Each prediction Pₙ from the neural network 24 is passed into the other main component of the controller 22, which is the prediction filter 26. One suitable prediction filter 26 is a Bayesian Filter (e.g., a Kalman Filter). The filter's internal state is the gait state, which changes and evolves over time as new measurements M and predictions Pₙ are received and processed. As schematically illustrated in FIG. 2, the filter 26 performs two steps, including a prediction step 32, and an update step 34.

In the prediction step 32, the filter 26 makes a prediction P_{f} of the current gait state separately from the prediction Pₙ of the primary neural network 32. This prediction P_{f} is generated using the previous gait state prediction Pₚ as the input, an internal process model 36 which predicts the values of P_{f} at the current time step by modelling how Pₚ evolved over the time between the current and previous time steps based on a physical or mechanical model of Pₚ, and a set of tunable gains 38.

In the update step 34, the filter 24 uses new measurements to update its own prediction P_{f} of the current gait state to the filtered prediction P′, which is the controllers current prediction of the current gait state. The filter 26 treats each prediction Pₙ of the primary neural network 24 as a measurement of the gait state directly.

Additionally, the filter 26 includes two other neural networks which are used in the update step 34. The first is a continuous data-driven gait model 40 regressed via a deep neural network and used to generate a prediction P_{kf} of the kinematic measurements M based on the filter's initial prediction P_{f} of the current gait state. The other is a heteroscedastic covariance model 28 that changes the filter's trust in the kinematic measurements M based on the filter's initial prediction P_{f} of the current gait phase and encodes prior knowledge about gait (e.g. foot angle is highly informative of incline during the stance phase of a gait cycle).

Further, to improve robustness, the filter 26 dynamically changes its trust in the direct measurements Pₙ of the gait state (i.e., the gait state predictions from the primary neural network 24) by generating another prediction Pₖₙ of the kinematics via the gait model 40—this time based on each gait state prediction Pₙ of the neural network 24—and then computing a Mahalanobis distance d_{M} between each prediction Pₖₙ and the actual live kinematic measurements M using the average covariance of the residuals from the gait model predictions. Thus, gait state predictions that yield incorrect kinematic predictions are distrusted.

The responsiveness of the controller 22 is easily tunable via a gain control 42 (e.g., Kalman gain), which allows for selective trust in both the kinematic measurements M, as encoded in the covariance output Cₖ of the covariance model 28, and the gait state predictions Pₙ from the primary neural network 24, as encoded in the Mahalanobis distance d_{M}. Additional description and functionality including information received by and information generated by several of the components of the controller architecture, are provided below.

The neural network 24 receives as an input the kinematic measurements M from the sensor(s) 20 and provides as an output a prediction Pₙ of the user's current gait state. A kinematic measurement M is any measurement related to the position, velocity, acceleration, or orientation of a natural or artificial part of the user's body. In this example, the kinematic measurement(s) may include foot angle, heel acceleration, etc. The gait state is a multi-parameter vector including parameters such as gait phase, walking speed, ground incline, stair locomotion, and/or starting or stopping motion, for example.

The gait phase is a parameter of the gait state indicating where the user is within a gait cycle and may be represented as a fraction of one gait cycle relative to an endpoint of the gait cycle. For example, the gait phase may have a value of zero at heelstrike (i.e., immediately following a leg swing phase) and may increase to a limit of 1 over a period during which the leg proceeds through an entire stance phase and an entire leg swing phase until the next heelstrike of the same leg. Some of the parameters of the gait state may themselves have values representing states rather than scalar quantities within a range of values. For example, a stair locomotion parameter may have a value of 1 for ascending stairs, −1 for descending stairs, or 0 for normal walking. A stopping motion parameter may be zero during locomotion and have a value of 1 when the gait state includes a stopping motion.

The neural network 24 is a machine learning model that functions as a feed-forward “black box” that has learned a relationship between the human gait state and kinematic measurements of the types provided by the sensor(s) 20, thus encoding that relationship. The prediction Pₙ is based on the learned relationship and is an effective decoding by the neural network 24 of the kinematic measurements M received from the sensor(s) 20. An example of a suitable neural network 24 is the Transformer machine learning model which can learn the relationship between kinematic measurements and gait state via inputs of wide ranges of data representing the relationship over a variety of different gait states from a variety of different human subjects, including able-bodied human subjects. For example, some large, open-source gait datasets are available to teach the neural network 24 this relationship. The relationship encoded by the neural network 24 can be further tuned to an individual user of the device 10 via further inputs of kinematic measurements taken during the user's own gait at multiple known gait states. It is contemplated that other existing or future-developed machine learning models may be adapted for use as the primary neural network 24.

The filter 26 receives as inputs: the gait state prediction Pₙ from the neural network 24, the kinematic measurements M from the sensor(s) 20, and the previous prediction Pₚ of gait state. The filter 26 provides as an output the filtered prediction P′ to be mapped to the output torque t using the torque profile 30. The output P′ is the controller's prediction of the current or n^{th} gait state, which is based at least in part on the filtered prediction P′ of the previous or (n−1)^{th} gait state. In other words, Pₚ=P′ₙ₋₁. As noted above, one suitable prediction filter 26 employs a Bayesian filter framework, such as a Kalman filter. Other similarly functioning filters are contemplated.

In the prediction step 32, the previous prediction Pₚ is the input, and the filter's initial prediction P_{f} of the current gait state is the output. In this step, the filter 26 applies the process model 36 to the previous prediction Pₚ to arrive at its initial prediction P_{f} of the current gait state. The process model 36 encodes knowledge on how the gait state naturally evolves over time. The speed at which the process model 36 evolves, and thus, the responsiveness of the filter 26, is tunable via a set of process gains 38.

The update step 34, the inputs are the filter's initial prediction P_{f} of the current gait state, the kinematic measurements M from the device sensor(s), and the gait state prediction Pₙ of the neural network 24. The output of the update step 34 is an updated prediction of the current gait state, as represented by P′. In this step, information about the likeliest gait state is extracted from the kinematic measurements M and combined with the predicted gait state P_{f} from the prediction step 32 to yield the updated gait state prediction P′. As is apparent in FIG. 2, several determinations, comparisons, and trust indicators are evaluated to arrive at P′.

As noted above, the continuous gait model 40 is used twice in the update step 34 to map a gait state vector as an input each time and return respective prediction Pₖ of the kinematics measurements. In one instance, the kinematics prediction P_{kf} based on the filter's initial prediction P_{f} of the current gait state is compared to the actual kinematic measurements M, and the filter 26 updates its initial gait state prediction P_{f} to correct for any discrepancy D_{kf} between predicted kinematics P_{kf} and measured kinematics M. In the other instance, the kinematics prediction Pₖₙ based on the initial prediction Pₙ of the current gait state from the neural network 24 is compared to the actual kinematic measurements M to determine the Mahalanobis distance d_{M} based on the discrepancy Dₖₙ between predicted kinematics Pₖₙ and measured kinematics M.

The covariance model 28 dynamically updates the trust in the kinematic measurements M received by the filter 26. This model uses as inputs the gait state prediction Pₙ from the neural network 24 and the filter's initial prediction P_{f} of the current gait state and returns the covariance Cₖ of the kinematic measurements M. This covariance output Cₖ encodes the level of trust associated with the kinematic measurements M. The covariance model can be tuned to shift the overall trust in the measurements M.

Another covariance model 44 dynamically updates trust in the predictions Pₙ of the gait state from the neural network 24 during the update step 34. This covariance model 44 uses as inputs the gait state prediction Pₙ from the neural network 24 and the kinematic measurements M and returns as an output the Mahalanobis distance d_{M}. Here, the gait model 40 is used to generate the prediction Pₖₙ of the kinematics based on the gait state prediction Pₙ from the neural network 24, which is then compared to the actual kinematics measurements M. A gait model covariance 46 is then used to compute the Mahalanobis distance d_{M} of the measured kinematics M from the predicted kinematics Pₖₙ and scales the trust in the gait state prediction Pₙ from the neural network 24 based on this distance.

The gait model covariance 40 is a matrix that scales the discrepancies Dₖₙ between the predicted kinematics Pₖₙ from the gait model 40 and the actual kinematics measurements M to have uniform ranges and is used to compute the Mahalanobis distance d_{M}. The Mahalanobis distance d_{M} encodes the distance between a point and a distribution and is an indicator of how unlikely a particular measurement is, given a prior distribution of those measurements. If the distance d_{M} is low—i.e. if the neural network 24 yields a kinematic prediction Pₖₙ that well-matches the actual measurement M—then the network gait state prediction Pₙ is likely correct and high trust is placed in the prediction Pₙ during the filter update step 34. Conversely, if the Mahalanobis distance d_{M} is high, then the network gait state prediction Pₙ is likely incorrect and is given a lower level of trust during the update step.

The gain control 42 applies a gain (e.g., a Kalman gain) to the discrepancy D_{kf} between the predicted kinematics P_{kf} and the measured kinematics M and to the discrepancy D_{g} between the filter's initial prediction P_{f} of the current gait state and the measured gait state (i.e., the gait state prediction Pₙ of the neural network 24). Depending on the respective levels of trust encoded by the covariance Cₖ of the kinematic measurements M and by the Mahalanobis distance d_{M}, each discrepancy may be resolved in favor of the more trusted side of the discrepancy to arrive at the filtered P′ prediction of the current gait state. For instance, if the Mahalanobis distance d_{M} is high, there is a relatively low level of trust in the current gait state prediction Pₙ from the neural network 24 and the discrepancy Dₖₙ may be resolved in favor of the filter's initial prediction P_{f} of the current gait state. The filter 26 thus acts as a check on the neural network's predictions Pₙ while continually implementing the neural network's predictions when they are more trustworthy than the filter's own predictions.

It is to be understood that the foregoing is a description of one or more embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A powered limb-assistive device comprising a controller that uses a neural network to generate an initial prediction of a current gait state of a user of the device based on kinematic measurements from the device and then filters the initial prediction to arrive at a current prediction of the current gait state of the user based at least in part on a continually updated measure of trust in the kinematic measurements.

2. The device of claim 1, wherein the neural network comprises a transformers machine learning model.

3. The device of claim 1, wherein the neural network has learned a relationship between the kinematic measurements and human gait states based at least in part on data obtained from human subjects other than the user.

4. The device of claim 3, wherein the learned the relationship is based at least in part on data obtained from the device while the user is using the device.

5. The device of claim 1, wherein the controller uses a Bayesian filter that generates an internal initial prediction of the current gait state based at least in part on a previous gait state prediction of the filter and updates the internal initial prediction to generate the current prediction of the current gait state of the user.

6. The device of claim 1, wherein the measure of trust is generated by another neural network that has learned a heteroscedastic covariance model that encodes prior knowledge about gait state parameters.

7. The device of claim 1, wherein the controller uses another neural network comprising a data-driven gait model used to generate a prediction of kinematic measurements for comparison with the kinematic measurements from the device.

8. The device of claim 1, wherein the gait state is a multiparameter vector comprising at least some of the following parameters: gait phase, walking speed, ground incline, a stair locomotion parameter, a stopping parameter.

9. A powered exoskeleton according to claim 1, comprising a joint and an actuator that applies a controlled torque at the joint based an output of the controller.

10. The powered exoskeleton of claim 9, wherein the joint is a leg joint and the exoskeleton assists user movement of a first portion of the leg relative to a second portion of the leg about the leg joint.

11. The powered exoskeleton of claim 10, wherein the joint is a knee joint or an ankle joint and the exoskeleton assists movement of a lower leg of the user relative to an upper leg of the user about the knee joint and/or assists movement of a foot of the user relative to the lower leg of the user about the ankle joint.
